(19) United States
(12) Patent Application Publication
(10) Pub. No.: US 2015/0230972 A1
(43) Pub. Date: Aug. 20, 2015

(54) THERMAL TISSUE CLOSURE DEVICE COMBINED WITH MECHANICAL VIBRATION

(71) Applicant: Calore Medical Ltd., Or Akiva (IL)

(72) Inventors: Giora Kornblau, Binyamina (IL); David Maier Neustadter, Nof Ayalon (IL)

(73) Assignee: Calore Medical Ltd.

(21) Appl. No.: 14/623,807

(22) Filed: Feb. 17, 2015

Related U.S. Application Data
(60) Provisional application No. 61/940,515, filed on Feb. 17, 2014.
(60) Provisional application No. 62/015,968, filed on Jun. 23, 2014.

(51) Int. Cl. A61F 7/00; A61B 17/00

(52) U.S. Cl. CPC A61F 7/00; A61B 17/0057; A61F 2007/0052; A61F 2007/0087

(57) ABSTRACT

A tissue closure device designed to reduce tissue adhesion during a thermal tissue closure procedure is described. The device comprises a heating tip, a support shaft, and a mechanical vibrator associated with the heating tip. The device uses mechanical vibration of the heating tip to reduce tissue adhesion to the heating tip during the thermal tissue closure procedure.

### CROSS-REFERENCES TO RELATED APPLICATIONS

This application claims priority to U.S. Provisional Application No. 61/940,515, filed Feb. 17, 2014, and U.S. Provisional Application No. 62/015,968, filed Jun. 23, 2014, which are incorporated herein by reference in their entirety.

### TECHNICAL FIELD

The present disclosure is directed towards the combination of vibration and heat in a tissue closure device, and more specifically, to a vibrating tissue closure device for closing a perforation or a puncture in a tissue region containing collagen.

### BACKGROUND

Heat may be used to shrink tissue containing collagen in various surgical and diagnostic procedures. For example, heat may be used to close and/or heal perforations or openings in tissue walls. For example, heat may be used to close arteriotomies on blood vessel walls by denaturing collagen within the tissue, thereby shrinking the tissue around the perforation/opening, and/or by inducing blood coagulation. In such procedures, heat may be generated using RF energy, ultrasound energy, laser energy, or conductive heating.

In any tissue heating application, there is a potential concern of tissue adhesion to the heating device, which can interfere with the proper functioning of the device. In thermal tissue closure applications, tissue adhesion to the heating device may interfere with heat conduction to the tissue site, affect the tissue closure process, and/or induce tissue damage or reopen the perforation in the tissue wall upon removal of the closure device.

Tissue adhesion in thermal treatment devices has traditionally been dealt with by coating the device tip with a non-stick coating. In devices that do not get hot themselves, e.g., ultrasonic surgical tools, active cooling of the device tip/tissue site is recommended to reduce tissue adhesion.

There remains a need for devices and mechanisms that reduce tissue adhesion during thermal treatment of tissues. In particular, devices and mechanisms designed to reduce tissue adhesion to a thermal tissue closure device are highly desirable.

### SUMMARY

The present disclosure is directed to a thermal tissue closure device that may use mechanical vibration of the heating tip to reduce tissue adhesion to the heating tip during a thermal tissue closure procedure.

It is to be understood that the terms “distal” and “proximal” are used in this disclosure with respect to a position or direction relative to the user, e.g., a physician, implementing the treatment procedure. “Distal” or “distally” are positions distant from or in a direction away from the user. “Proximal” or “proximally” are positions near or in a direction towards the user.

One application of the devices and methods of the present disclosure is the thermal closure of puncture sites (arteriotomies) on blood vessel walls. The particular requirements of small vessel size, precision in closing arteriotomies, quick and efficient implementation to reduce unnecessary stress on the patient, etc., may, in some circumstances, make tissue adhesion particularly challenging for thermal vascular closure procedures. The present disclosure describes devices and methods that address the potential challenges of tissue adhesion for thermal vascular closure. The disclosed devices and methods are not limited to blood vasculature applications, and may be applied to any vessel, duct, cavity, and/or tissue tract found in the body.

Consistent with an exemplary embodiment of the present disclosure, a device for controlled shrinkage of a tissue region containing collagen is described. The device may include a heating tip configured to deliver heat to the tissue region, a support for positioning the heating tip at a location to effect delivery of heat to the tissue region, and a mechanical vibrator associated with the heating tip. The heating tip, the vibrator, and the support may be configured to cooperate to facilitate controlled shrinkage of the tissue region.

Other aspects of this disclosure are contained in the accompanying drawing, description, and claims. Thus, this summary is exemplary only, and is not to be considered restrictive.

The accompanying drawing, which is incorporated in and constitutes a part of this specification, illustrates an embodiment and together with the description, serves to explain the principles of the various aspects of the embodiment.

### BRIEF DESCRIPTION OF DRAWING

The accompanying drawing, which is incorporated in and constitutes a part of this specification, illustrates an embodiment and together with the description, serves to explain aspects of the embodiment.

FIG. 1 illustrates a tissue closure device, in accordance with exemplary embodiments of the present disclosure;

It is to be understood that both the foregoing general description and the following detailed description are exemplary and explanatory only and are not restrictive of the claims.

### DESCRIPTION OF EXEMPLARY EMBODIMENTS

Reference will now be made to certain embodiments consistent with the present disclosure, an example of which is illustrated in the accompanying drawing. It is to be understood that the device of the present disclosure can be employed to close or heal perforations in any tissue region of a patient's body, or to generally cause shrinkage of tissue in any part of a patient's body.

The present disclosure describes a tissue closure device that combines heat and mechanical vibration to effect controlled shrinkage of tissue containing collagen. Exemplary embodiments may include a conductive heating tip configured to deliver heat to a tissue region containing collagen. In such a device, when electric current passes through a resistive element, heating of the conductive heat tip occurs. In other embodiments, the heating element may include any electrical, chemical, mechanical, or other mechanism for causing heat, such as, for example, ultrasound heating, RF heating, laser heating, microwave heating, inductive heating, or any other local tissue heating technology.

The heating tip may be made of any material capable of resulting in heat, including metal, ceramics, composites, polymers, or combinations thereof. Similarly, the heating tip may be structured in any way and be capable of causing the desired heating. Exemplary shapes may include spherical, semi-spherical, dome-shaped, and any other shape or construction capable of producing the desired heat. The heating tip may be flexible, allowing its shape to adapt to the shape of the tissue being heated and/or allowing the tip to be inserted through a hole smaller than the resting diameter of the tip. The heating tip may be further encased in a housing or coating designed to limit tissue adhesion, but otherwise enabling the resulting heat to be directed to a target tissue area. The heating tip may include one or more heating elements configured to generate heat when energy (e.g., electrical energy) is directed to the heating element.

Disclosed embodiments may also include a support for positioning the heating element at a location to effect delivery of heat to the tissue region. The support may include any structure that enables positioning of the heating element adjacent target tissue. Thus, depending on the target tissue at issue, the support may assume alternative shapes or configurations. By way of example, the support may be an elongate element. The particular shape and construction of an elongated support may vary, and might include, by way of example only, one or more of a tube, rod, shaft, bar, rib, or column. In exemplary embodiments, the support may further define a conduit or channel for directing energy to the heating element. By way of example, the conduit may be used to provide electrical connection between the heating tip and a power source provided in a handle of the tissue closure device.

Embodiments may also include a mechanical vibrator associated with the heating tip. As used herein, the term “associated with” includes any relationship where the mechanical vibrator causes vibration of the heating tip. In exemplary embodiments, the mechanical vibrator may be a high frequency mechanical vibrator. The mechanical vibrator may be configured to induce high frequency vibration of the heating tip to reduce tissue adhesion to the heating tip both during and after a thermal tissue closure procedure. Other potential benefits of the high frequency vibration may include the reduction of friction as the heating tip is inserted into the tissue tract, and enhancement of the heat conductance from the heating tip to the tissue due to better mechanical contact between the tip and the tissue. In some embodiments, vibration of the heating tip may also provide tactile feedback to the user (i.e., the physician) regarding the thermal tissue closure procedure.

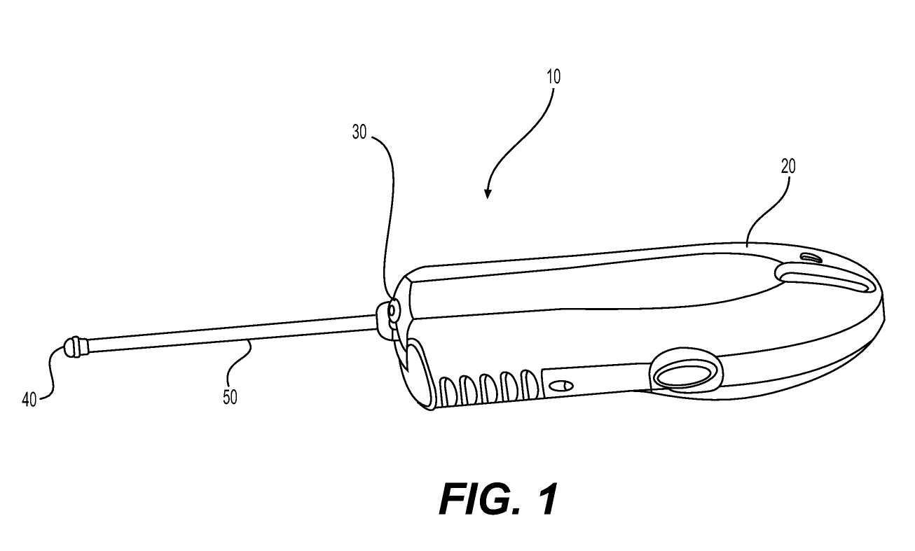

FIG. 1 shows an exemplary embodiment of a tissue closure device 10 comprising a handle 20, a heating tip 40, a mechanical vibrator 30, and a support shaft 50 connecting handle 20 to heating tip 40. The size and configuration of heating tip 40 may be selected to facilitate thermal contact between heating tip 40 and the target tissue region, and enhance heat conduction between heating tip 40 and the target tissue region. In exemplary embodiments, heating tip 40 may receive energy from the power source via an electrical conductor that runs through support shaft 50. The power source may provide electrical energy to heating tip 40 to heat the tissue region, and thereby effect shrinkage of collagen-containing tissue to close or heal a perforation or an opening in the tissue region.

In some embodiments, as shown in FIG. 1, mechanical vibrator 30 may be mounted on handle 20 at the proximal end of support shaft 50. In other embodiments, mechanical vibrator 30 may be mounted within handle 20. In yet other embodiments, mechanical vibrator 30 may be mounted on or within heating tip 40. Although FIG. 1 illustrates a single mechanical vibrator 30, some embodiments may include multiple mechanical vibrators 30 associated with heating tip 40. In embodiments where mechanical vibrator 30 is mounted on or within handle 20, support shaft 50 may comprise a conduit or lumen (not shown) for directing the vibrations to heating tip 40.

In an exemplary embodiment, mechanical vibrator 30 may be an electric vibration motor. In another embodiment, mechanical vibrator 30 may be an ultrasound transducer. In yet another embodiment, mechanical vibrator 30 may be an audio frequency speaker.

In exemplary embodiments, mechanical vibrator 30 may have a set amplitude of vibration. In other embodiments, mechanical vibrator 30 may have a variable amplitude of vibration. In such embodiments, the physician may select the amplitude of vibration of heating tip 40 prior to the treatment process. In some embodiments, the physician may be able to change the amplitude of vibration during the treatment process. In exemplary embodiments, the amplitude of vibration of heating tip 40 may be between about 0.1 μm and 100 μm. In another embodiments, the amplitude of vibration of heating tip 40 may be between about 50 μm and about 100 μm. In yet another embodiment, the amplitude of vibration of heating tip 40 may be between about 100 μm and about 300 μm.

In exemplary embodiments, mechanical vibrator 30 may have a set frequency of vibration. In other embodiments, mechanical vibrator 30 may have a variable frequency of vibration. In such embodiments, the physician may select the frequency of vibration of heating tip 40 prior to the treatment process. In some embodiments, the physician may be able to change the frequency of vibration during the treatment process. In exemplary embodiments, the frequency of vibration of heating tip 40 may be between about 10 Hz and about 1 kHz, or between about 100 Hz and about 20 kHz, or between about 20 kHz and about 60 kHz, or between about 50 kHz and about 1 MHz, or between about 500 kHz and 20 MHz.

Exemplary embodiments may also include at least one processor within handle 20. The processor may be configured to access information related to vibration of heating tip 40 and to regulate mechanical vibrator 30. In one embodiment, the processor may regulate the amplitude and frequency of vibration of mechanical vibrator 30. As used herein, the term “processor” may include an electric circuit that performs a logic operation on input or inputs. For example, such a processor may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations. The at least one processor, may be included in or may be coincident with, for example, a control system configured to regulate delivery of energy from a power source to heating tip 40. In one embodiment, the control system may regulate the delivery of energy to heating tip 40 in accordance with a temperature profile.

In exemplary embodiments, the duration of vibration of mechanical vibrator 30 may be regulated by the processor. In some embodiments, heating tip 40 may vibrate throughout the treatment procedure. In such embodiments, heating tip 40 may be set to vibrate from before the insertion of tissue closure device 10 into the patient's body until after the removal of the device. In some embodiments, mechanical vibrator 30 may be set to vibrate when heating tip 40 is actively heated. In some other embodiments, mechanical vibrator 30 may be set to vibrate for a predetermined period of time during the treatment process. In such embodiments, when the predetermined time threshold is reached, the processor may automatically shut off mechanical vibrator 30. In some other embodiments, the processor may automatically shut off mechanical vibrator 30 when the heater associated with heating tip 40 is turned off. In yet another embodiment, the processor may automatically shut off mechanical vibrator 30 following a predetermined cool-down time after the heater is turned off.

In some embodiments, mechanical vibrator 30 may be configured to follow a preselected vibration profile. In such an embodiment, the processor may regulate the duration, amplitude, and frequency of vibration of mechanical vibrator 30 based on the preselected vibration profile.

In some exemplary embodiments, the control system provided in handle 20 may include a closed loop system, coupling a temperature sensor to the processor configured to regulated mechanical vibrator 30. In such an embodiment, the temperature of the tissue region or heating element 40 may be monitored by the processor, and mechanical vibrator 30 may be regulated by the processor based on the sensed temperature of the tissue region and/or heating tip 40. For example, in some embodiments, mechanical vibrator 30 may be set to start vibration when heating tip 40 and/or the tissue region reaches a predetermined temperature. In some other embodiments, mechanical vibrator 30 may be set to stop vibration when heating tip 40 and/or the tissue region reaches a predetermined temperature. In another embodiment, mechanical vibrator 30 may be set to follow a predefined heating profile, i.e., mechanical vibrator 30 may be set to start and stop vibration according to a predetermined temperature of heating tip 40 and/or the tissue region. In some embodiments, a physician operating tissue closure device 10 may be permitted to override the closed loop system, if desired.

In exemplary embodiments, vibration of heating tip 40 may serve as a tactile indicator to the user of the progress of the treatment process. For example, in some embodiments, vibration of heating tip 40 may indicate to the user that heating of the tissue region is in progress. In some other embodiments, cessation of vibration of heating tip 40 may serve as a tactile indicator to the user that tissue closure device 10 may be removed from the patient.

Other embodiments will be apparent to those skilled in the art from consideration of the specification and practice of the embodiments disclosed herein. It is intended that the specification and examples be considered as exemplary only, with a true scope and spirit of the embodiments being indicated by the following claims.

1. A device for controlled shrinkage of a tissue region containing collagen, the device comprising:
a heating tip configured to deliver heat to the tissue region;
a support for positioning the heating tip at a location to effect delivery of heat to the tissue region; and
a mechanical vibrator associated with the heating tip;
wherein the heating tip, the mechanical vibrator, and the support are configured to cooperate to facilitate controlled shrinkage of the tissue region.

2. The device of claim 1, further comprising a handle connected to the support.

3. The device of claim 2, wherein the mechanical vibrator is positioned within the handle.

4. The device of claim 2, wherein the mechanical vibrator is positioned in the handle at the proximal end of the support.

5. The device of claim 1, wherein the mechanical vibrator is positioned in the heating tip.

6. The device of claim 1, wherein the mechanical vibrator is an ultrasound transducer.

7. The device of claim 1, wherein the mechanical vibrator is an audio frequency speaker.

8. The device of claim 1, wherein the mechanical vibrator is a vibration motor.

9. The device of claim 1, wherein the mechanical vibrator is a high frequency mechanical vibrator.

10. The device of claim 1, wherein the mechanical vibrator is configured to provide tactile feedback to a user.

11. The device of claim 1, further comprising a processor configured to receive temperature feedback from a temperature sensor associated with the heating tip and to regulate vibration of the mechanical vibrator based on the temperature feedback.

12. The device of claim 11, wherein the processor is configured to regulate at least one of an amplitude, a frequency and a duration of vibration of the mechanical vibrator.

13. The device of claim 1, wherein the mechanical vibrator is configured to vibrate at a frequency between about 10 Hz and about 1 kHz.

14. The device of claim 1, wherein the mechanical vibrator is configured to vibrate at a frequency between about 100 Hz and about 20 kHz.

15. The device of claim 1, wherein the mechanical vibrator is configured to vibrate at a frequency between about 20 kHz and about 60 kHz.

16. The device of claim 1, wherein the mechanical vibrator is configured to vibrate at a frequency between about 50 kHz and about 1 MHz.

17. The device of claim 1, wherein the mechanical vibrator is configured to vibrate at a frequency between about 500 kHz and about 20 MHz.

18. The device of claim 1, wherein the mechanical vibrator is configured to vibrate at an amplitude of between about 0.1 μm and about 100 μm.

19. The device of claim 1, wherein the mechanical vibrator is configured to vibrate at an amplitude of between about 50 μm and about 200 μm.

20. The device of claim 1, wherein the mechanical vibrator is configured to vibrate at an amplitude of between about 100 μm and about 300 μm.
